(19) United States
(12) Patent Application Publication
(10) Pub. No.: US 2023/0052517 A1
(43) Pub. Date: Feb. 16, 2023

(54) ELECTROCHEMICAL SENSING METHODS AND APPARATUS FOR DETERMINING DRUG UPTAKE AND RETENTION IN CELLS

(71) Applicant: The University of Manitoba, Winnipeg (CA)

(72) Inventors: Sabine Kuss, Winnipeg (CA); Tran Le Huy Luu, Winnipeg (CA)

(73) Assignee: The University of Manitoba, Winnipeg, MB (CA)

(21) Appl. No.: 17/790,581

(22) PCT Filed: Dec. 31, 2020
(86) PCT No.: PCT/CA2020/051811

§ 371 Date: Jul. 1, 2022

Related U.S. Application Data
(60) Provisional application No. 62/957,109, filed on Jan. 3, 2020.

(51) Int. Cl. G01Q 60/60; C12M 3/06; C12M 1/42; G01N 27/30

(52) U.S. Cl. CPC G01Q 60/60; C12M 23/16; C12M 35/02; G01N 27/30

(57) ABSTRACT

This invention provides methods and apparatuses for the rapid assessment of cell permeability by a drug. More particularly, described herein a method of determining membrane permeability (influx and/or efflux) of a cell to a drug, the method including: (a) obtaining a biological sample; (b) dispersing at least one cell from the biological sample to a discrete location; (c) exposing the at least one cell to one member of a drug panel in a drug solution, wherein the drug panel is composed of drugs of a given concentration; (d) incubating the at least one cell from the biological sample in the drug for a given time; (e) obtaining at least one electro-analytical measurement of the discrete location adjacent the at least one cell.

### CROSS REFERENCE TO RELATED APPLICATION

This application claims the benefit of U.S. Provisional Patent Application Ser. No. 62/957,109 filed on 3 Jan. 2020, entitled “ELECTROCHEMICAL SENSING METHODS AND APPARATUS FOR DETERMINING DRUG UPTAKE AND RETENTION IN CELLS”.

### FIELD OF THE INVENTION

This invention relates to electrochemical sensing of antibiotics and anti-cancer drugs to evaluate membrane permeability of a target cell to the drug. More particularly, the invention relates to the determination of drug resistance in a cell from a biological sample using electrochemistry.

### BACKGROUND

The rapid spread of drug resistance in bacteria as well as cancer has developed into a significant threat to the global public health.¹ According to the World Health Organization (WHO), antibiotic resistance is present in every country,² and various national and international health organizations, including the United Nations, the Infectious Diseases Society of America, as well as the Public Health Agency of Canada, have called for the urgent development of new treatment and diagnostic strategies.^{3,4} The Centers for Disease Control and Prevention reports approximately 10 million deaths worldwide each year in connection with antibiotic resistance.⁵ Similarly, drug resistance in cancer is believed to be responsible for treatment failure in up to 90% of metastatic cancer patients.⁶ Cellular resistance mechanisms in both bacteria and cancer include cell membrane protein modifications, intracellular drug target alterations, and the over expression of efflux pumps.⁷⁻⁹ The overexpression of efflux pump proteins enable cells to expel drugs rapidly from the cell interior, before these compounds can take effective action.⁸ New methodologies to understand and detect drug resistance in both bacteria and cancer by electrochemistry are under development.

In recent years, the innovation of electrochemical sensors has attracted immense attention, due to their high sensitivity, rapid analysis and ability to analyze complex samples, such as urine and blood. Although no sensor for the point-of-care detection of antibiotic resistance has been proposed so far, electrochemical sensors have become a powerful tool in various fields, such as environmental monitoring¹⁰, biotechnology¹¹, and industrial process control^{12,13}. Electrochemical sensors are fast, sensitive, cost effective, and allow for direct in vitro analysis of analytes in biological samples without much preparation. Accordingly, electrochemistry is very attractive for its use in medical applications¹⁴ and a number of research articles have emerged over the last decade that represent attempts at the analysis of a drug by electrochemistry. For example, electrochemistry was used for the assessment of curcumin on the viability of human glioblastoma cells by measuring the electrochemical signals (Epc=−0.05 V vs. Ag/AgCl), obtained with cyclic voltammetry⁴³, whereby the electrochemical signal is not attributed to cell membrane permeability, but shows that the electrochemical signal decreases when the cells die and no electrochemistry of curcumin is shown in the paper.

Simultaneous electrochemical detection of both anti-cancer drugs ifosfamide (IFO) and etoposide (ETO) by cyclic voltammetry (CV) and differential pulse voltammetry (DPV)⁴² was shown, whereby the authors modify electrodes to detect Ifosfamide and Etoposide simultaneously. However, at no time were living biological samples tested (i.e. no cells nor bacteria) and drugs were measured in solution (i.e. urine and blood serum) or immobilized at electrodes. Nevertheless, these experiments showed that the electrodes had low detection limits and could detect drug concentration changes.

Similarly, electrochemical sensing of Oxaliplatin was undertaken in biological samples. The authors modify electrodes to reach very low detection limits of Oxaliplatin and tested for interference by other drugs, but no cell studies were carried out⁴⁴.

### SUMMARY OF THE INVENTION

This invention relates to antibiotics and anti-cancer drugs for use evaluating membrane permeability of a target cell to the drug. Alternatively, the drugs may be electro-active antibiotics and electro-active anti-cancer drugs. In order to detect drug resistance by electrochemistry, suitable target analytes have been identified and their interaction with biological cells have been characterized. In particular, characterization of antibiotics and anti-cancer drugs that are electro-active are useful in identifying antibiotics and anti-cancer drugs that are most suitable for administration to a target cell, whereby electrochemical analysis of a target cell can provide useful information about possible drug resistance based on drug permeability measurements (i.e. influx and efflux). However, non-electro-active antibiotics and non-electro-active anti-cancer drugs may also be detected using electrochemical analyses. Furthermore, such analysis may also useful for the design and development of novel pharmaceare is based on the surprising discovery that quantitative electrochemical measurements of antibiotics and anti-cancer drugs in vitro can reliably predict drug resistance by a target cell as a representation of the drugs permeability of the target cell.

In a first aspect, there is provided a method of determining membrane permeability of a cell to a drug, the method including: (a) obtaining a biological sample; (b) dispersing at least one cell from the biological sample to a discrete location or attached to a discrete substrate; (c) exposing the at least one cell to one member of a drug panel in a drug solution, wherein the drug panel is composed of drugs of a given concentration; (d) incubating the at least one cell from the biological sample in the drug for a given time; (e) obtaining at least one electro-analytical measurement of the discrete location adjacent the at least one cell.

The method may further include exchanging the drug solution for a drug-less solution. The method may further include further incubating the at least one cell from the biological sample in the drug-less solution for a given time. The method may further include at least one further electro-analytical measurement of the discrete location adjacent to the at least one cell. The drug may be an electro-active drug. The drug may be selected from: an antibiotic drug and an anticancer drug. The drug may be selected from: an electro-active antibiotic drug and an electro-active anticancer drug. The antibiotic drug may be selected from one or more of the following: ampicillin; penicillin; amoxicillin; neomycin; tobramycin; ciprofloxacin; levofloxacin; norfloxacin; enrofloxacin; ofloxacin; linezolid; tetracycline; and azithromycin. Alternatively, the antibiotic may be a Tobramycin-Ciprofloxacin hybrid compound. The anticancer drug may be selected from one or more of the following: 6-mercaptopurine; 5-fluorouracil; gemcitabine; doxorubicin; mitoxantrone; epirubicin; daunorubicin; valrubicin; cisplatin; temodal; oxaliplatin; carboplatin; etoposide; ifosfamide; erlotinib; irinotecan; and roscovitine. The drug panel may be comprised of multiple drugs each at a variety of concentrations or combinations of drugs each combination at a variety of concentrations. The drug panel may be comprised of multiple electro-active drugs each at a variety of concentrations or combinations of electro-active drugs each combination at a variety of concentrations. The biological sample may include bacteria isolated from a patient. The biological sample may include a cancer biopsy from a patient. The electro-analytical measurement may be made by one or more of the following: linear sweep voltammetry (LSV); cyclic voltammetry (CV); differential pulse voltammetry (DPV); differential pulse anodic stripping voltammetry (DPASV); square wave voltammetry (SWV); adsorptive stripping linear sweep voltammetry (AdSLSV); electrochemical impedance spectroscopy (EIS); chronoamperometry (CA); chronopotentiometry (CP); chronocoulometry (CC); impact chemistry (IC); scanning ion conductance microscopy (SICM); scanning electrochemical cell microscopy (SECCM); scanning photoelectrochemical microscopy (SPECM); and scanning electrochemical microscopy (SECM). The electro-analytical measurement may be made by one or more of the following: cyclic voltammetry (CV); electrochemical impedance spectroscopy (EIS); impact chemistry (IC); and scanning electrochemical microscopy (SECM). The electro-analytical measurement may be made by one or more of the following: linear sweep voltammetry (LSV); cyclic voltammetry (CV); differential pulse voltammetry (DPV); differential pulse anodic stripping voltammetry (DPASV); square wave voltammetry (SWV); adsorptive stripping linear sweep voltammetry (AdSLSV); electrochemical impedance spectroscopy (EIS); chronoamperometry (CA); chronopotentiometry (CP); chronocoulometry (CC); and impact chemistry (IC). The electro-analytical measurement may be made by cyclic voltammetry (CV). The electro-analytical measurement, may be made by impact chemistry (IC). The electro-analytical measurement, may be made by scanning electrochemical microscopy (SECM). The electrode, may be optimized for the electro-active drug or electro-active drugs at the discrete location.

In a further aspect, there is provided, an apparatus, the apparatus including (a) cell retention array having a plurality of array locations; and (b) a corresponding electrode array, wherein each electrode corresponds to each array location or a group of electrode locations and wherein the electrode is selected to be operable for a corresponding drug solution.

In a further aspect, there is provided a microfluidic device, the microfluidic device including (a) a plurality of cell retention locations; and (b) a corresponding electrode for each cell retention location or locations and wherein the electrodes are selected to be operable for a corresponding drug solution which might be delivered to the retention location or locations. The microfluidic device may further include a system for fluid exchange at one or more of the retention locations.

In a further aspect, there is provided an apparatus, the apparatus including (a) a plurality of cell retention substrates; and (b) a corresponding electrode associated with each cell retention substrate, wherein the electrode is selected to be operable for a corresponding drug solution. The cell retention substrates may be beads.

In an alternative embodiment, the bacterial cells or other cells, may be made to collide with a metal wire electrode or other electrode in impact chemistry (IC), whereby the electrode provides an electro-analytical measurement of the bacterial cells or other cells with which the metal wire electrode or other electrode collides.

In an alternative embodiment, an apparatus is provided that includes a cell retention array having a plurality of array locations; wherein each cell retention array location corresponds to an electrode, and wherein each electrode is suitable for deposition of a cell on the surface of the electrode.

In an alternative embodiment, an apparatus is provided that includes an array of electrodes operable to retain at least one cell on the surface of the electrode, wherein the electrodes are distributed at a plurality of electrode array locations.

Each electrode may be operable to retain the cell on the electrode by dropcasting. Furthermore, each electrode may be operable to receive a drug solution. Dropcasting is the result of depositing of an aqueous cell solution, containing a cell, on an electrode, whereby when the aqueous cell solution evaporates, it leaves the cells “sticking” to the electrode without killing the cell, such that the cells' internal composition and osmotic pressure is not compromised.

In an alternative embodiment, a method is provided for determining membrane permeability of a cell to a drug, the method including: (a) obtaining a biological sample; (b) dispersing at least one cell from the biological sample to a surface or attached to a surface; (c) exposing the at least one cell to a drug solution, wherein the drug solution has a given drug concentration; (d) incubating the at least one cell from the biological sample in the drug solution for a given time; (e) obtaining at least one electro-analytical measurement of the at least one cell by impact chemistry (IC), whereby the at least one cell from the biological sample is made to collide with an electrode. The electrode may be a metal wire electrode.

### BRIEF DESCRIPTION OF THE DRAWINGS

FIG. 1 shows electrochemical quantification of CP efflux from living cells. (A) When expelled from the cell, CP is oxidized at the electrode during SECM. The resulting current signal increased with increasing CP efflux (B). (C) During impact chemistry cells in solution collide with a wire electrode whereby the CP diffusion layer around the cells (pink) is oxidized at the electrode. This will provide statistical data over populations of cells. (D) shows a schematic for electrochemical detection of drug efflux from a Pseudomonas bacteria.

FIG. 2 shows electrochemical characterization of carboplatin (CP), where in (A) CP exhibits an oxidation peak at 0.8 V vs Ag/AgCl reference electrode; (B) at unmodified platinum electrodes a limit of detection (LOD) of 50 μM was found; (C) a pH dependency study revealed that CP can be detected at a pH range of 1 to 7.5; and in (D) shows a schematic of electrochemical drug efflux studies in Pseudomonas bacteria. Bacteria (diagonal arrow) are drop-casted onto a macro-electrode. When expelling ciprofloxacin, the antibiotic is electrochemically oxidized at the electrode, resulting in a current increase seen as peak during DPV.

FIG. 3 shows a schematic representation of DR in bacteria, wherein the membrane protein modification, drug target alteration, drug inactivation by intracellular enzymes, and membrane efflux pumps can prevent drugs to enter and/or affect the cell.

FIG. 4 shows antibiotic hybrids for electrochemical investigations, with (A) Structure of the tobramycin-ciprofloxacin hybrid, containing a 12-carbon-long aliphatic (C12) hydrocarbon linker and (B) Cyclic voltammetry of 2 mM tobramycin-ciprofloxacin hybrid at various scan rates.

FIG. 5 shows a schematic representation of SECM for biological applications. (A) Instrumental design, including Z-axis positioner (I), constant distance controller (II), light source (III), electrochemical cell (IV), as well as working electrode (WE), counter electrode (CE) and reference electrode (RE). (B) Example of a microelectrode and its size comparison (C) as well as top view (D) of the same electrode. (E) Representation of the low current bi-potentiostat, connected to all three electrodes.

FIG. 6 shows a schematic representation of electrochemical measurements on living bacteria. (A) Bacteria dropcasted onto a macroelectrode and exposed to an antibiotic (A), which is expelled by efflux pump from the organism. The antibiotic is then electrochemically converted at the electrode. (B) SECM electrode scanning across small islands of bacteria, crossing DR bacteria, as well as non-resistant entities. (C) Schematic of an expected current profile of lateral scan across living bacteria

FIG. 7 shows cell patterning of HeLa cells using elastomeric through-hole membranes. (A) Photograph of a through-hole membrane and its middle part (B). Insets showing SEM images of a top (A) and side view (B). Scale bars: 500 μm. (C, D, E) Cell patterns achieved for HeLa in island sizes of 400 μm (C), 200 μm (D) and 50 μm (E). Scale bars: 100 μm. F) Optical micrograph of E. coli patterns in 20 μm islands.

FIG. 8 shows a schematic representation of resistance adaptation monitored by SECM. (A) Fluorescently labelled DR and non-DR bacteria immobilized in co-culture will be imaged by an SECM microelectrode, resulting in a 3D current intensity map (B).

FIG. 9 shows the peak current recorded at various scan velocities of the microelectrode, wherein the initial electrochemical response recorded prior to carboplatin exposure for both carboplatin-susceptible (A2780-s) and carboplatin-resistant (A2780-cp) ovarian cancer cells and the slope of the linear regression was shows the cells' ability to regenerate FcCH₂OH through the cellular export of glutathione, to indicate stress experienced by the cells due to carboplatin.

FIG. 10 shows Ciprofloxacin (1 mM) uptake quantification in both resistant and sensitive Pseudomonas aeruginosa bacterial strains using differential pulse voltammetry (DPV).

FIG. 11 shows Tobramycin (2 mM) uptake quantification in both resistant and sensitive Pseudomonas aeruginosa bacterial strains using differential pulse voltammetry (DPV).

FIG. 12 shows Ciprofloxacin-Tobramycin (Cip-Tob) hybrid influx quantification in P. aeruginosa by DPV.

FIG. 13 shows electrochemical detection of ciprofloxacin export from Pseudomonas bacteria.

FIG. 14 shows electrochemical detection of ciprofloxacin export in PAO1 and PA262 Pseudomonas bacterial strains.

### DETAILED DESCRIPTION OF THE INVENTION

The following detailed description, will be better understood when read in conjunction with the appended figures. For the purpose, of describing the invention, the figures demonstrate embodiments of the present invention. However, the invention is not limited to the precise arrangements and examples shown.

As used herein a “drug” refers to any therapeutic moiety, which includes small molecules and biological agents (for example, proteins, peptides, nucleic acids). As used herein, the term drug may in certain embodiments include any therapeutic moiety, or a subset of therapeutic moieties. For example, but not limited to one or more of the potentially overlapping subsets and one or more drugs, as follows: antibiotic drugs; and anticancer drugs.

As used herein an “electro-active drug” refers to any molecule that can produce detectable electro-activity, and which, also has therapeutic activity. The molecular structure is the primary determinant of a compound's electro-activity, whereby the presence of particular functional groups (for example, phenol, aromatic amine, thiol, nitro, nitrophenol and quinone groups) and/or whether the structure permits for delocalization of a positively or negatively changed group. In particular, the electro-activity of a given drug compound may be based on the oxidation-reduction (redox) potential of the compound, or whether the compound is prone to undergo an oxidation-reduction reaction by gaining or losing an electron.

As used herein “membrane permeability” refers to the influx and/or efflux of an electro-active drug into or out of a cell. Depending on the electro-active drug and the cell, there may be by passive diffusion, facilitated passive diffusion, active transport, and pinocytosis. Similarly, once a drug is within a given cell, the drug may be removed from the cell by an efflux pump or other cell transport mechanism.

As used herein a “drug panel” refers a panel of drugs or electro-active drugs of various concentrations selected based on the target cell or cells being tested. For example, where the target cell is a cancer cell, then the panel would be made of anti-cancer drugs and these drugs may be tested at a variety of concentrations, such that an at least one cell deposited at a discrete location may be incubated with a member of the drug panel. Similarly, where the target cell is a bacterial cell, then the panel would be made of antibacterial drugs and these drugs may be tested at a variety of concentrations, such that an at least one cell deposited at a discrete location may be incubated with a member of the drug panel. When we look at impact chemistry cells (both cancer and bacteria) the cells may be in solution, where the cells are governed by Brownian motion colliding with an electrode. For example, this could be also implemented in a microfluidic device with a solution that may be exchanged or added to, leaving the at least one cell at the discrete location.

Anti-cancer drugs may be categorized as alkylating agents (bi and monofunctional), anthracyclines, cytoskeletal disruptors, epothilone, topoisomerase inhibitors (I and II), kinase inhibitors, nucleotide analogs and precursor analogs, peptide antibiotics, platinum-based agents, Vinka alkaloids, and retinoids. Alkylating agents, may be bifunctional alkylators (for example, Cyclophosphamide, Mechlorethamine, Chlorambucil and Melphalan) or monofunctional alkylators (for example, Dacarbazine(DTIC), Nitrosoureas and Temozolomide). Examples of anthracyclines are Daunorubicin, Doxorubicin, Epirubicin, Idarubicin, Mitoxantrone, and Valrubicin. Cytoskeletal disruptors or taxanes are Paclitaxel, Docetaxel, Abraxane and Taxotere. Epothilones may be epothilone or related analogs. Histone deacetylase inhibitors may be Vorinostat or Romidepsin. Inhibitors of topoisomerase I may include Irinotecan and Topotecan. Inhibitors of topoisomerase II may include Etoposide, Teniposide or Tafluposide. Kinase inhibitors may be selected from Bortezomib, Erlotinib, Gefitinib, Imatinib, Vemurafenib or Vismodegib. Nucleotide analogs and precursor analogs may be selected from Azacitidine, Azathioprine, Capecitabine, Cytarabine, Doxifluridine, Fluorouracil, Gemcitabine, Hydroxyurea, Mercaptopurine, Methotrexate or Tioguanine/Thioguanine. Peptide antibiotics like Bleomycin or Actinomycin. Platinum-based agents may be selected from Carboplatin, Cisplatin or Oxaliplatin. Retinoids may be Tretinoin, Alitretinoin or Bexarotene. The Vinca alkaloids and derivatives may be selected from Vinblastine, Vincristine, Vindesine and Vinorelbine. Alternatively, an electro-active anti-cancer drug may be selected from TABLE 1.

TABLE 1
Anti-cancer drug detection by electrochemistry.

| | | Electrode | Method of | |
|---|---|---|---|---|
| Classes | Drug | modification | analysis | LOD [M] |
| Antimetabolite | 6-Mercaptopurine | MWCNT Paste | LSV | 1 × 10⁻⁷ |
| | | electrode | | |
| Antimetabolite | 6-Mercaptopurine | [Co(phen)3]3+-GO- | DPV | 1.5 × 10⁻⁸ |
| | | dsDNA/GCE | | |
| Antimetabolite | 6-Mercaptopurine | N-HCNS-Pd-MIP/ | DPASV | 7.2 × 10⁻¹⁰ |
| | | IL-PGE | | |
| Antimetabolite | 5-Fluorouracil | Glucose/CPE | CV, DPV | 5.17 × 10⁻⁹ |
| Antimetabolite | 5-Fluorouracil | BMPA/Flexible AuE | CV, SWV | 3.4 × 10⁻⁷ |
| Antimetabolite | 5-Fluorouracil | Reduced | CV, SCV, | 1.24 × 10⁻⁹ |
| | | GO-CS/GCE | SWV | |
| Antimetabolite | 5-Fluorouracil | AuNP-MWCNT- | CV, DPV | 2.0 × 10⁻⁸ |
| | | CS/ GCE | | |
| Antimetabolite | 5-Fluorouracil | AuNP-PFR/CPE | CV, DPV | 6.70 × 10⁻⁷ |
| Antimetabolite | 5-Fluorouracil | PANINT-AgNP/PGE | DPV | 6 × 10⁻⁸ |
| Antimetabolite | 5-Fluorouracil | IL/CPE | CV, DPV | 1.3 × 10⁻⁸ |
| Antimetabolite | 5-Fluorouracil | GO-MWCNT/GCE | CV, SWV | 1.6 × 10⁻⁸ |
| | | and SPCE | | |
| Antimetabolite | 5-Fluorouracil | CuSAE | CV, | 1.2 × 10⁻⁹ |
| | | | AdSLSV | |
| Antimetabolite | 5-Fluorouracil | MTB/CPE | CV, DPV | 2.04 × 10⁻⁹ |
| Antimetabolite | Gemcitabine | AuE | DPV | 6 × 10⁻⁸ |
| Antimetabolite | Gemcitabine | MMOF-AuNP/AuE | LSV | 3 × 10⁻¹⁵ |
| Cytotoxic | Doxorubicin | MAb-AuNP-TBSol- | EIS | 1.7 × 10⁻¹³ |
| antibiotic | | Gel/AuE | | |
| Cytotoxic | Doxorubicin | Mab-AuNP- | EIS | 3.1 × 10⁻¹² |
| antibiotic | | APTES/SSE | | |
| Cytotoxic | Doxorubicin | Pd@PtNP-MWCNT/ | AdSSWV | 8.6 × 10⁻¹⁰ |
| antibiotic | | GCE | | |
| Cytotoxic | Mitoxantrone | dsDNA-MWCNT- | DPV | 1.3 × 10⁻⁸ |
| antibiotic | | AgNP-PTP/GCE | | |
| Cytotoxic | Epirubicin | Ag-MWCNT/GCE | SWV, CV | 1.0 × 10⁻⁹ |
| antibiotic | | | | |
| Cytotoxic | Daunorubicin | N-rGO-SWCNT- | DPV | 5.7 × 10⁻⁹ |
| antibiotic | | PtNP/GCE | | |
| Cytotoxic | Valrubicin | AuNP-EDA- | CV | 1.8 × 10⁻⁸ |
| antibiotic | | MWCNT/AuE | | |
| Alkylating | Cisplatin | GST/CPE | CV, SWV | 8.8 × 10⁻⁶ |
| agents | | | | |
| Alkylating | Cisplatin | MWCNT/SPCE | CV, DPV | 4.6 × 10⁻⁶ |
| agents | | | | |
| Alkylating | Temodal | dsDNA-AuNP/PGE | DPV | 1.0 × 10⁻⁹ |
| agents | | | | |
| Inhibitors | Etoposide | Au-Pd@rGO-L- | DPV | 7.18 × 10⁻¹⁰ |
| | Ifosfamide | Cysteine/PGE | | |
| Inhibitors | Erlotinib | MWCNT-PUFIX- | DPV | 2 × 10⁻⁸ |
| | | PPHF/PGE | | |
| Inhibitors | Irinotecan | GCE | CV | 1.12 × 10⁻¹⁰ |
| Inhibitors | Roscovitine | PGE or SPCE | SWV | PGE: 1.96 × 10⁻⁷ |
| | | | | SPCE: 1.53 × 10⁻⁷ |

([Co(phen)3]3+ = cobalt (III) trisphenanthroline complex; BMPA = biopolymer from babassu mesocarp modified with phthalic anhydride; PFR = porphyran; PANINT = polyaniline nanotube; CuSAE = Copper solid amalgam electrode; AdSLSV = adsorptive stripping linear sweep voltammetry; Pd@PtNP = mesoporous Palladium and Platinum Core shell nanoparticles; AdSSWV = adsorptive stripping square wave voltammetry; PTP = polythiophene; N-rGO = nitrogen-doped reduced graphene oxide; GST = Glutathione-s-transferase; Au-Pd@rGO = gold, palladium and reduced graphene oxide nanocomposite; PUFIX = polyurethane; PPHF = polypropylene hollow fiber).

An anti-cancer drug that may be used as described herein, may be selected from one or more of: Actinomycin; All-trans retinoic acid; Azacitidine; Azathioprine; Bleomycin; Bortezomib; Carboplatin; Capecitabine; Cisplatin; Chlorambucil; Cyclophosphamide; Cytarabine; Daunorubicin; Docetaxel; Doxifluridine; Doxorubicin; Epirubicin; Epothilone; Etoposide; Fluorouracil; Gemcitabine; Hydroxyurea; Idarubicin; Imatinib; Irinotecan; Mechlorethamine; Mercaptopurine; Methotrexate; Mitoxantrone; Oxaliplatin; Paclitaxel; Pemetrexed; Teniposide; Tioguanine; Topotecan; Valrubicin; Vemurafenib; Vinblastine; Vincristine; Vindesine; and Vinorelbine. Alternatively, the anti-cancer drug may be a biological agent and may be selected from Herceptin (Trastuzumab), Ado-trastuzumab, Lapatinib, Neratinib, Pertuzumab, Avastin, Erbitux or radiolabelled antibodies or targeted radiotherapies such as PSMA-radioligands. The anti-cancer drug may be an Androgen Receptor, an Estrogen Receptor, epidermal growth factor receptor (EGFR) antagonists, or tyrosine kinase inhibitor (TKI). An anti-angiogenesis agent may be selected from avastin, an epidermal growth factor receptor (EGFR) antagonists or tyrosine kinase inhibitor (TKI). An Immune modulator such as Bacillus Calmette-Guerin (BCG). Alternatively, an anti-cancer drug may include hybrids of two or more of the preceding anti-cancer drugs.

Alternatively, an electro-active antibiotic drug may be selected from TABLE 2.

TABLE 2
Antibiotic drug detection by electrochemistry.

| | | Method of | | Electrode |
|---|---|---|---|---|
| Classes | Drug | analysis | LOD [M] | modification |
| B-Lactams | Ampicillin | DPV | 3.2 × 10⁻¹¹ | dsDNA/AMP aptamer |
| B-Lactams | penicillin | CV | 8 × 10⁻¹⁶ | RGO/AuNP |
| B-Lactams | penicillin | CV | 1.05 × 10⁻⁵ | multisegment nanoparticles |
| B-Lactams | Amoxicillin | CV | 6 × 10⁻⁷ | POT(SDS) |
| B-Lactams | Amoxicillin | CV | 5 × 10⁻⁶ | Ni/CR |
| B-Lactams | Amoxicillin | SWV | 9 × 10⁻⁶ | AuNP-PdNP-RGO |
| B-Lactams | Amoxicillin | SWV | 1.2 × 10⁻⁷ | CB DHP |
| B-Lactams | Amoxicillin | CV | 1.87 × 10⁻⁹ | poly acridine orange |
| Aminoglycosides | Neomycin | SWV | 1.07 × 10⁻⁶ | Polyamic acid/GO |
| Aminoglycosides | Tobramycin | CV | 1.4 × 10⁻¹⁰ | Polypyrrole |
| Quinolones | Ciprofloxacin | CV, ASV | 5.9 × 10⁻⁸ | Graphene |
| Quinolones | Ciprofloxacin | CV, DPV | 5 × 10⁻⁸ | CTAB |
| Quinolones | Ciprofloxacin | CV | 1.2 × 10⁻⁸ | MgFe2O4-MWCNT |
| Quinolones | Ciprofloxacin | CV, LSV | 9 × 10⁻⁷ | MWCNT |
| Quinolones | Ciprofloxacin | CV | | GO |
| Quinolones | Ciprofloxacin | CV | 3.3 × 10⁻⁶ | BDD |
| Quinolones | Ciprofloxacin | SWV | 3.3 × 10⁻⁸ | GCP |
| Quinolones | Ciprofloxacin | CV | 6 × 10⁻⁶ | MWCNT |
| Quinolones | Levofloxacin | DPV | 1 × 10⁻⁶ | PoAP/MWCNT |
| Quinolones | Levofloxacin | CV, SWV | 1.4 × 10⁻⁸ | AgNPs-CB-PEDOT:PSS |
| Quinolones | Levofloxacin | CV, DPV | 5.3 × 10⁻⁷ | MIP/G-AuNPs |
| Quinolones | Levofloxacin | CV, SWV | 2.88 × 10⁻⁶ | BDD |
| Quinolones | Levofloxacin | CV | 1 × 10⁻⁸ | AgNP |
| Quinolones | Norfloxacin | SWV | 3.4 × 10⁻⁸ | Polyamic acid/GO |
| Quinolones | Norfloxacin | LSV | 5 × 10⁻⁸ | MWCNT |
| Quinolones | Enrofloxacin | LSV | 5 × 10⁻⁷ | MWCNT |
| Quinolones | Ofloxacin | CV | 1.8 × 10⁻¹⁰ | MWCNT |
| | | SW-AdAsV | 2.4 × 10⁻¹⁰ | |
| Quinolones | Ofloxacin | CV, DPV | 1 × 10⁻⁹ | AuNPs/ATP/ABA |

RGO = reduced graphine oxide; POT (SDS) = poly(o-toluidine) (sodium dodecyl sulphate); CB = carno black; DHP = dihexadecylphosphate; CTAB = cetyltrimethylammonium bromide; BDD = boron doped diamond; GCP = glassy carbon paste; PoAP = poly(o-aminophenol); PEDOT:PSS = poly(3,4-ethylenedioxythiophene)-poly(styrenesulfonate); G = graphene; ATP = 4-aminothiophenol; ABA = 4-aminobenzoic acid (4-ABA); IL-G = ionic liquid- graphene; ZSM = mesoporous zeolitic material.

An antibiotic drug that may be used as described herein, may be selected from one or more of: Amikacin; Gentamicin; Kanamycin; Neomycin; Netilmicin; Tobramycin; Paromomycin; Streptomycin; Spectinomycin(Bs); Geldanamycin; Herbimycin; Rifaximin; Carbacephem; Loracarbef; Carbapenems; Ertapenem; Doripenem; Imipenem/Cilastatin; Meropenem; Cefadroxil; Cefazolin; Cephradine; Cephapirin; Cephalothin; Cefalexin; Cefaclor; Cefoxitin; Cefotetan; Cefamandole; Cefmetazole; Cefonicid; Loracarbef; Cefprozil; Cefuroxime; Cefixime; Cefdinir; Cefditoren; Cefoperazone; Cefotaxime; Cefpodoxime; Ceftazidime; Ceftibuten; Ceftizoxime; Moxalactam; Ceftriaxone; Cefepime; Ceftaroline fosamil; Ceftobiprole; Teicoplanin; Vancomycin; Telavancin; Dalbavancin; Oritavancin; Clindamycin; Lincomycin; Lipopeptide; Daptomycin; Clarithromycin; Erythromycin; Roxithromycin; Telithromycin; Spiramycin; Fidaxomicin; Aztreonam; Nitrofurans; Furazolidone; Nitrofurantoin(Bs); Linezolid; Posizolid; Radezolid; Torezolid; Amoxicillin; Ampicillin; Azlocillin; Dicloxacillin; Flucloxacillin; Mezlocillin; Methicillin; Nafcillin; Oxacillin; Penicillin G; Penicillin V; Piperacillin; Penicillin G; Temocillin; Ticarcillin; Amoxicillin/clavulanate; Ampicillin/sulbactam; Piperacillin/tazobactam; Ticarcillin/clavulanate; Bacitracin; Colistin; Polymyxin B; Enoxacin; Gatifloxacin; Gemifloxacin; Levofloxacin; Lomefloxacin; Moxifloxacin; Nadifloxacin; Nalidixic acid; Norfloxacin; Ofloxacin; Trovafloxacin; Grepafloxacin; Sparfloxacin; Temafloxacin; Sulfacetamide; Sulfadiazine; Silver sulfadiazine; Sulfadimethoxine; Sulfamethizole; Sulfamethoxazole; Sulfanilimide; Sulfasalazine; Sulfisoxazole; Trimethoprim-Sulfamethoxazole (Co-trimoxazole) (TMP-SMX); Sulfonamidochrysoidine; Demeclocycline; Doxycycline; Metacycline; Minocycline; Oxytetracycline; Tetracycline; Clofazimine; Dapsone; Capreomycin; Cycloserine; Ethambutol(Bs); Ethionamide; Isoniazid; Pyrazinamide; Rifampicin; Rifabutin; Rifapentine; Streptomycin; Arsphenamine; Chloramphenicol(Bs); Fosfomycin; Fusidic acid; Metronidazole; Mupirocin; Platensimycin; Quinupristin/Dalfopristin; Thiamphenicol; Tigecycline(Bs); Tinidazole; and Trimethoprim(Bs). Alternatively, an antibiotic drug may include hybrids of two or more of the preceding antibiotic drugs. For example, an antibiotic hybrid molecule described herein is tobramycin-ciprofloxacin (Tob-Cip).

As used herein “electro-analytical measurement” may be obtained by one or more of the following: linear sweep voltammetry (LSV); cyclic voltammetry (CV); differential pulse voltammetry (DPV); differential pulse anodic stripping voltammetry (DPASV); square wave voltammetry (SWV); adsorptive stripping linear sweep voltammetry (AdSLSV); electrochemical impedance spectroscopy (EIS); chronoamperometry (CA); chronopotentiometry (CP); chronocoulometry (CC); impact chemistry (IC); scanning ion conductance microscopy (SICM); scanning electrochemical cell microscopy (SECCM); scanning photoelectrochemical microscopy (SPECM); and scanning electrochemical microscopy (SECM).

As used herein “dropcasting” is meant to describe the pipetting of or otherwise depositing of an aqueous cell solution, such as bacteria or cancer cell, on an electrode, whereby when the aqueous cell solution evaporates, it leaves the cells “sticking” to the electrode without killing the cell, such that the cells' internal composition and osmotic pressure is not compromised. Accordingly, dropcasting is alternative method for putting a functional cell in contact with an electrode, whereby the cells are not actually dried, just the aqueous cell solution surrounding the cells that is being evaporated.

### Materials and Methods

### Electrochemistry

Standard electrochemical methods, such as cyclic voltammetry (CV), and scanning electrochemical microscopy (SECM), may be used. However, the methodology could be adapted to use other methods such as linear sweep voltammetry (LSV), differential pulse voltammetry (DPV), differential pulse anodic stripping voltammetry (DPASV), square wave voltammetry (SWV), adsorptive stripping linear sweep voltammetry (AdSLSV), electrochemical impedance spectroscopy (EIS), chronoamperometry (CA), chronopotentiometry (CP), chronocoulometry (CC), impact chemistry (IC), scanning ion conductance microscopy (SICM) or scanning electrochemical cell microscopy (SECCM). SECM has been successfully utilized to study drug resistance in mammalian cancer cells¹⁵⁻¹⁸. In particular, applying SECM to drug resistant cancer cells as compared to non-drug resistant cells, showed different electrochemical behaviours¹⁵. Furthermore, use of cell permeable and impermeable redox mediators that allowed the extraction of kinetic information from experimental SECM data, which resulted in the quantification of drug resistance on the single cell level by mathematical and numerical models¹⁶. In addition, it has been demonstrated that SECM may be used to assess cancer cells, exposed to antioxidants, and their electrochemical response over time may be quickly acquired. Also, it was possible to determine a samples' apparent heterogeneous rate constant, independent from their topography, which until this point remained a challenge to the SECM community¹⁷⁻¹⁸.

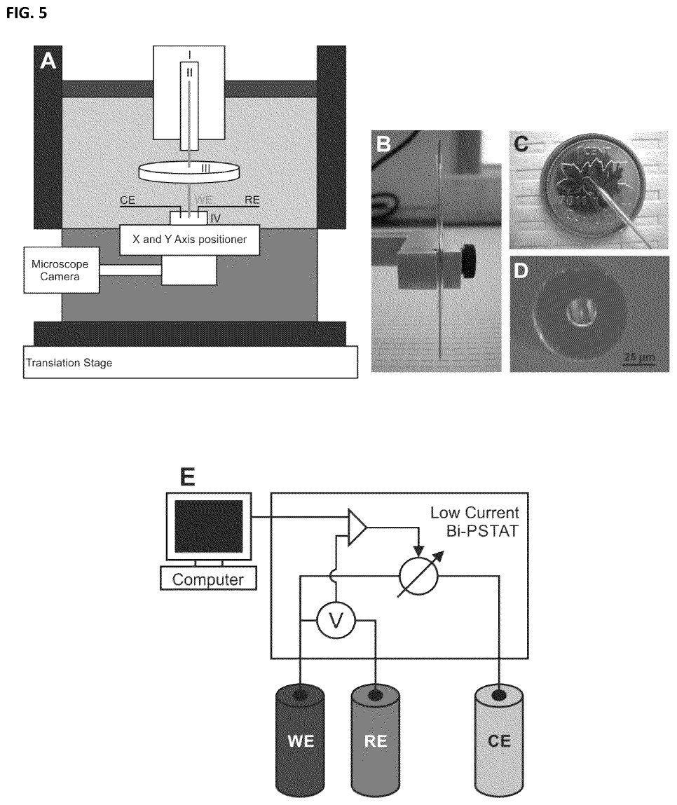

These successful studies on cancer cells present an ideal basis for the investigation of antibiotic resistance in bacteria. Similar to cancer cells, the electrochemical response of bacteria is based on the expression of membrane efflux pumps, which affect drug take-up and release from the organisms and subsequent local and transported concentration of electroactive material to the electrode. Scanning electrochemical microscopy (SECM) is an electroanalytical technique, employing a micro- or nanoscale electrode, which is rastered across a surface to analyse its electrochemical activity. As shown in FIG. 5, a microelectrode (FIG. 5A orange wire, B, C, and D) consists of a metal wire, which is sealed into a quartz capillary and is connected to a potentiostat¹⁹. This electrode functions as working electrode (WE) and is mounted onto a motor station, moving in the z-direction above an electrochemical cell, which is mounted onto an X and Y axis positioner²⁰. An incorporated light source and microscope, equipped with a camera, allows the monitoring of any sample during electrode positioning prior to the data acquisition, as well as during SECM measurements²¹. Sample observation becomes especially important when working with biological samples, such as living bacteria or tissue cells, as the target's morphology can be observed during the experiment. Furthermore, the SECM apparatus is placed on a vibration isolation table inside a Faraday cage to avoid interference from external electric noise.

In the presence of a redox active species in solution, a potential, far exceeding the standard potential of the dissolved redox mediator, can be applied at the WE to drive the oxidation or reduction of a redox species at the surface of the electrode. A potentiostat compares the potential difference between WE and a reference electrode (RE) to a computer defined value and adjusts a power source between WE and a counter electrode (CE) accordingly (FIG. 5E). Thereby, a current commonly in the fA to μA range is measured at the WE. In fact, some redox mediators have been shown to interact with biological entities (e.g. living bacteria or tissue cells)²²⁻²³, and many biological samples have been successfully analysed in the past by SECM²⁴.

Cyclic Voltammetry (CV) is an electrochemical technique in which an applied potential is swept linearly between two limiting potentials, driving a chemical reaction at macro-, micro- or even nanoelectrodes²⁵. The overall CV shape of a redox reaction at electrodes is determined by, and provides information about, redox thermodynamics, electron transfer kinetics, diffusion processes of molecules in solution and towards the electrode, and possible decay reactions²⁵.

Bioelectrochemical sensors have attracted immense attention, whereby electrode materials and electrode modifications have emerged for the design of highly sensitive and selective sensors.²⁶ Electroanalytical techniques are cost effective, sensitive and the transparency of a solid or liquid sample is irrelevant, allowing direct in vitro analysis of food, beverage, blood, urine, and saliva samples and tissue samples with minimal preparation using electroanalytical methods, such as cyclic voltammetry (CV), chronoamperometry (CA), impact chemistry and scanning electrochemical microscopy (SECM), to quantify electro-active drug compounds released from cells.

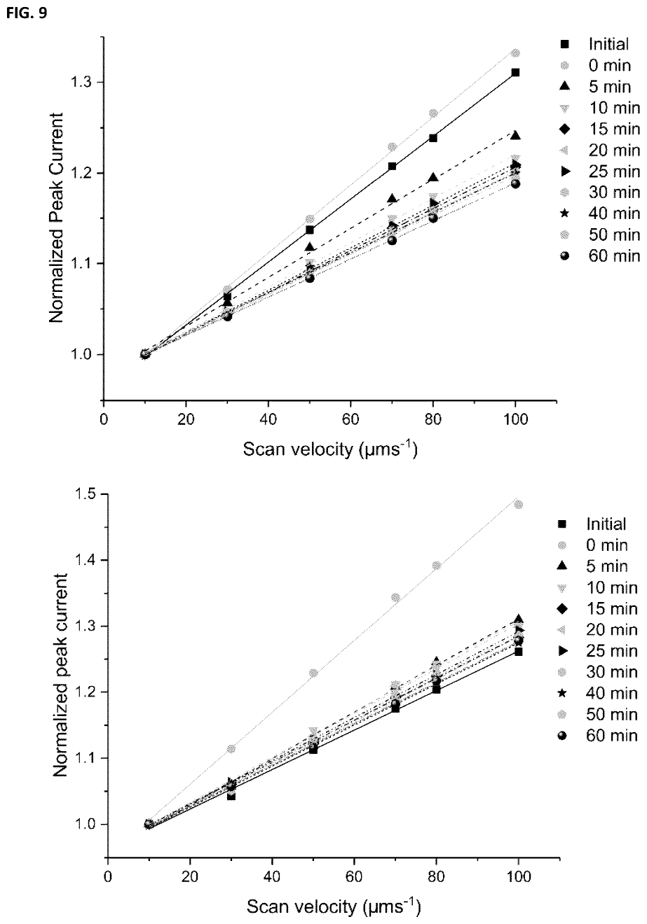

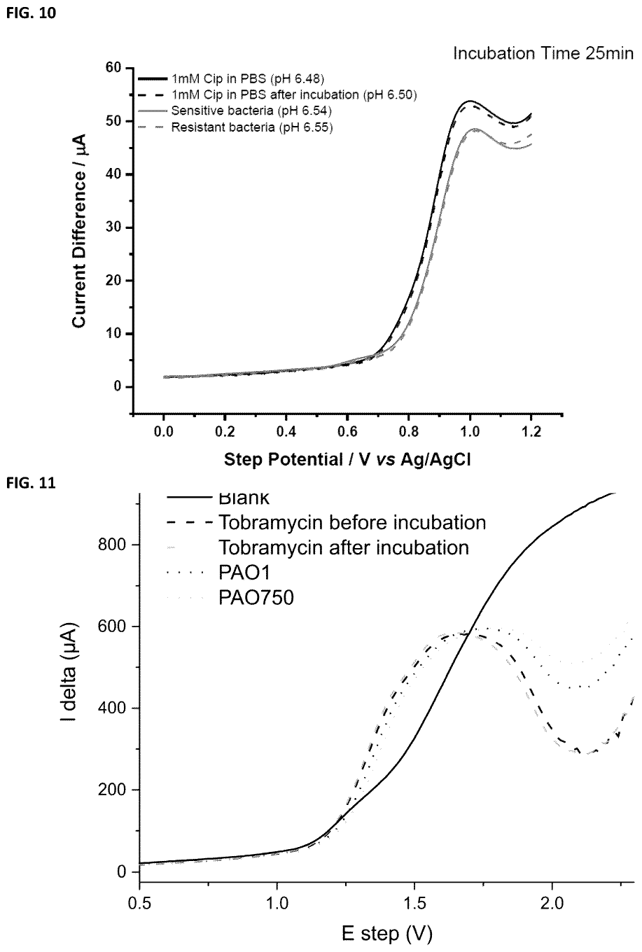

The bioelectrochemical studies of Pseudomonas aeruginosa as shown in FIG. 10 utilized differential pulse voltammetry (DPV), and four (4) glass vials were prepared, holding a PBS solution containing 1 mM ciprofloxacin. Vial 1 was used as a control; Vial 2 was a second control with ciprofloxacin after 25 minutes of incubation at 37° C. These two controls demonstrate that the incubation at 37° C. does not lead to a degradation of ciprofloxacin in solution; Vials 3 and 4 each contain P. aeruginosa cells at a cell number range of 10⁶ to 10⁸ per ml held for 25 minutes at 37° C. DPV measurements of 1 mM Cip were taken and after incubation, centrifugation was performed at 4000 rpm for 10 min and the supernatant was collected. Any ciprofloxacin taken up by the bacteria during the incubation time was hence removed from the solution. DPV measurements were then performed on the supernatant as an indication of ciprofloxacin uptake by the bacteria.

Similarly uptake of Tobramycin (2 mM), was analyzed in Pseudomonas aeruginosa also using DPV. The control solutions were tested before and after incubation at 37° C. and with different incubation times at 37° C. (i.e. 15 minutes; 30 minutes and 60 minutes). Two Pseudomonas aeruginosa bacterial strains were analyzed for drug uptake (PAO1 strain—a wild type strain, containing efflux pumps on the bacterial membranes; and PAO750—a hyper-susceptible strain where efflux pumps have been deleted). Integration underneath the curves provides the charge. This charge is proportional to the number of molecules transferred during DPV recordings (see FIGS. 11-14).

### Ovarian Cancer Cell Studies

Carboplatin-susceptible (A2780-s) and carboplatin-resistant (A2780-cp) ovarian cancer cells were grown in petri dishes at 37° C. until a confluence of approximately 60 to 70% was reached. Cells were exposed to a PBS solution containing 1 mM ferrocenemethanol (FcCH₂OH) at 37° C. for 45 minutes. Following this incubation, the petri dish was inserted into a scanning electrochemical microscope (SECM), equipped with a camera, fluorescence unit and a heating stage. Target cells were identified using an approach curve and a horizontal line scan was carried out across the cells every 5 to 10 minutes. The solution in the petri dish was exchanged for a PBS solution containing 2 mM carboplatin and 1 mM ferrocenemethanol (FcCH₂OH). Horizontal line scans were carried out every 5 to 10 minutes to record the electrochemical response by the cells.

Electrochemical response was recorded prior to drug exposure (i.e. carboplatin) for both carboplatin-susceptible (A2780-s) and carboplatin-resistant (A2780-cp) ovarian cancer cells and the slope of the linear regression was shown to be related to the cells' ability to regenerate FcCH₂OH through the cellular export of glutathione, wherein the extend of FcCH₂OH regeneration by the cells can be expressed as apparent heterogeneous rate constant^{17, 46}.

### Electrodes

A three-electrode setup may be used for cyclic voltammetry (CV) and scanning electrochemical microscopy (SECM) or other electrochemical analyses. Electrodes may have a 25 micrometer platinum (Pt) diameter or laser pulled Pt working electrodes, an Ag/AgCl (3 M NaCl) pseudo-reference electrode (calibrated in FcCH₂OH) and 0.5 mm Pt auxiliary. The preparation of conventional 25 micrometer Pt microelectrodes followed a well-established fabrication protocol⁴⁰ while polished; needle-like, disk-shaped nanoelectrodes were fabricated using a similar to the procedures described⁴¹. The fabrication procedure specifically produces disk shaped Pt microelectrode sealed in a quartz capillary and laser pulled until a dimensionless radius of glass (RG) inferior to 10 is obtained. In brief, 25 μm annealed Pt wires were pulled into quartz glass capillaries (length of 150 mm, an outer diameter of 1 mm, and an inner diameter of 0.3 mm) under vacuum with the help of a P-2000 laser pipet puller (Sutter Instruments™, CA, USA). The pulling program results in the formation of a long and sharp microelectrode with a thin glass sheath, which facilitates membrane penetration. The effective radius was evaluated from steady-state voltammetry. Electrodes with diameters < and/or >25 um may be used. For example, 10 μm, 5 μm, 1 μm in diameter or even on the nanoscale). Marcoeletrodes (diameter 1 mm) may be used for voltammetric measurements. Alternatively, a metal wire may be used as an electrode (for example, in impact chemistry).

### Statistical Analysis

All values were measured in triplicates and subsequently statistically evaluated. Based on a student's t-distribution, errors were calculated applying a two-tailed test with n=3, α=0.025 and therefore a confidence level (CL) of 95% is given.

### EXAMPLES

### Example 1. Electrochemical Analysis of Anticancer Drug Carboplatin (CP)

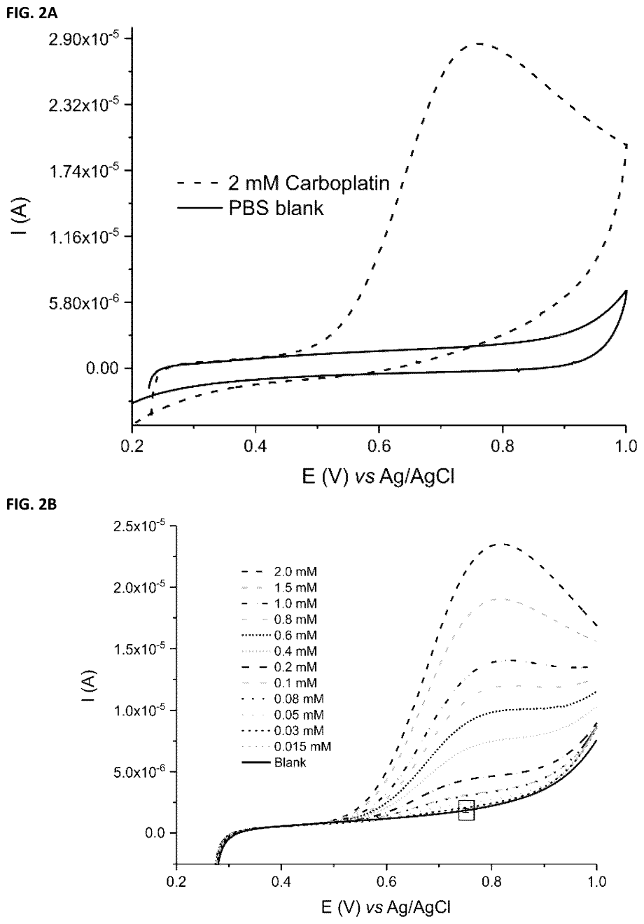

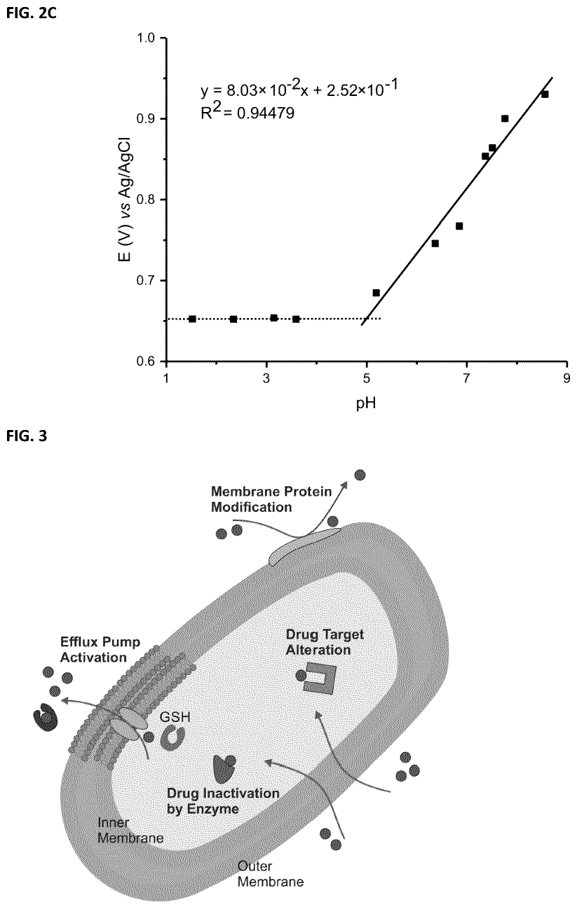

Cyclic voltammetry (CV) is an electrochemical technique in which an applied potential is swept linearly between two limiting potentials, driving a chemical reaction at macro-, micro- or even nano-electrodes. The overall CV shape of a reduction/oxidation (redox) reaction at the electrodes is determined by, and provides information about, redox thermodynamics, electron transfer kinetics, diffusion processes of molecules in solution and towards the electrode, and possible decay reactions.²⁵ Furthermore, experiments have determined an oxidation peak of carboplatin at 0.8 V vs Ag/AgCl reference electrode (FIG. 2A). The oxidation of carboplatin (CP) was found to be irreversible and no electrode blockage by its oxidation reaction products was observed. A detection limit (LOD) of 50 μM at unmodified platinum electrodes was identified (FIG. 2B), whereby CP can be detected at a pH range of 1 to 7.5 (FIG. 2C). This characterization shows that CP can be recognized electrochemically at low concentrations and its diffusion in solution is understood.

In the literature, nanoparticles have been successfully used to increase the surface area of electrodes to ultimately lowering the necessary overpotential applied to drive the oxidation/reduction reaction.²⁶ To increase the sensitivity of CP detection at platinum (Pt) electrodes, platinum nanoparticles (PtNPs) may be drop-casted onto electrodes as a first approach. The current recorded at the electrode is expected to rise with increasing concentration of PtNPs. An optimal concentration of PtNPs, may be determined to enable the detection of CP or another anti-cancer electroactive drug at sub-μm concentrations. However, during live-cell imaging using the scanning probe technology SECM, dropcasted PtNPs are unlikely to be stable at the electrode surface during scans. Hence, a mixture of PtNPs and pyrrole will be polymerized at the electrode surface using CV. This may result in a stable conductive polymer layer, capturing PtNPs. The porous nature of polypyrrol may allow for efficient electron transfer and diffusion of CP towards the electrode. The thickness of the polymer layer can be controlled by adjusting the duration of polymerization.

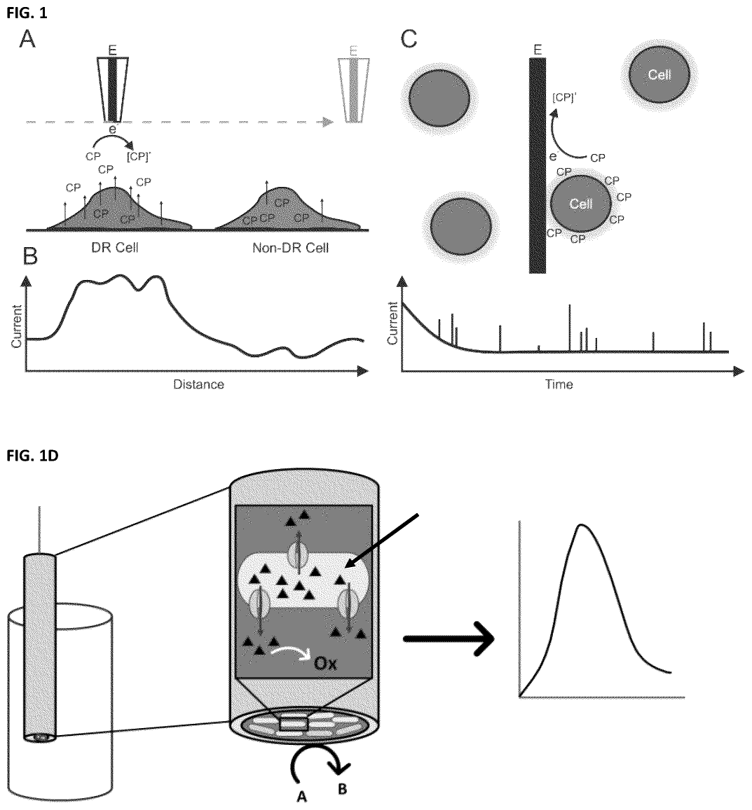

Electrochemical quantification of carboplatin efflux from A2780 endometrioid EOC cell lines was tested. Paired, syngeneic A2780 EOC cells that are chemosensitive (A2780-s) or chemosresistant (A2780-cp). Cells may be patterned in defined areas on the surface of plastic substrates using elastomeric through-hole membranes.¹⁶ The applicability of these substrates has been demonstrated in the past for HeLa cells. Cell patterning is useful for Bio-SECM studies, as target cells can be easily located through the SECM-integrated optical microscope and cells will not be able to “crawl” away during repeated measurements. When cells are patterned on plastic, a microelectrode may be brought in close proximity to the A2780 cells using the SECM setup to maximize the recorded current response. For this purpose, an approach curve over or next to a monolayer of cells will be carried out in the presence of a redox mediator, which is cell impermeable and will have no influence on the biological sample of interest. Hexaammineruthinium (III) chloride is the substance of choice based on literature¹⁶ to carry out such an approach curve. The probe may be retracted to any desired distance above the cells (for example, 10 μm). A2780 cells may be grown at 37° C. in cell growth medium containing CP. Concentration and time of incubation may be optimized. CP may be removed by exchanging the solution to fresh growth medium without CP. The SECM electrode may be moved horizontally across an island or single cells while recording the electrochemical current, resulting from the oxidation of CP that is expelled by the cells (FIG. 1A). This allows for a comparison of cells positioned side-by-side of different resistance phenotypes at the same time and under the same conditions. Cells of higher magnitude of drug resistance (higher efflux rate) are expected to result in higher current values during SECM measurements (FIG. 1B). Due to the sensitivity of the SECM methodology it may be possible to tell the exact number of CP molecules exported from a single cell per second. This will provide a numerical measure for the drug resistant phenotype for any cell of interest. To obtain statistical data on a large number of cells, impact chemistry may be used. Impact chemistry is a powerful technique for the detection of single biological entities in large numbers.^{38, 39} Impact chemistry is based on faradaic charge transfer, following the collision of redox active entities on the nano- or micrometer scale with an electrode. Cells pre-exposed to CP may be put into fresh cell medium lacking serum. Governed by Brownian motion, single cells would collide with the electrode, which may be held at an oxidizing or reducing potential. Collision events will result in the oxidation CP released from single cells, revealing a short current burst (“spike”) every time a cell passes the electrode, whereby the spike intensity is related to the drug efflux rate.

While A2780-s and A2780-cp cells may be employed, different EOC cell lines representing most histotypes, as well as EOC patient cell samples may also be tested. Cells will be measured and compared for their resistance phenotype by SECM. Patient samples may be used to determine the cells susceptibility against the panel of electroactive anti-cancer drugs and at various concentrations. Testing patient samples may allow for personalized clinical management.

Further work was done by the inventors⁴⁵ to evaluate the electrochemical detection of chemotherapeutic uptake by ovarian cancer cells, whereby an electrochemical characterization of carboplatin was evaluated for detection limits and pH dependence. Furthermore, bioelectrochemical studies quantified carboplatin uptake by ovarian cancer cells. Voltammetric drug uptake studies demonstrated the detection of carboplatin uptake in one carboplatin-susceptible and one carboplatin-resistant ovarian cancer cell line.

The electrochemical response of ovarian cancer cells to carboplatin was assessed by scanning electrochemical microscopy. In A2780-cp cells, an increase in slope right after carboplatin exposure was observed and the increase relaxes back to its initial value within 5 to 10 minutes. It is hypothesized that this electrochemical response indicates the ability of resistant cells to cope with the exposure to carboplatin by temporarily increasing the rate of glutathione efflux, transporting not only glutathione, but also carboplatin to the cell exterior (FIG. 9). A clear difference between carboplatin-susceptible and carboplatin-resistant cells was observed by SECM measurements.

### Example 2. Electrochemical Analysis of Anti-Biotic Drugs

The ability of biological entities, such as bacteria, to remain unaffected by at least one antimicrobial agent is referred to as drug resistance (DR), whereby the non-susceptibility to 1 agent in antimicrobial categories is called multidrug resistance. DR can be due to the acquisition of genes encoding for defence mechanisms to a specific agent or to overexpression of efflux pumps, which can rapidly expel drugs from the cell interior. Membrane protein modification, drug target alteration, drug inactivation by bacterial enzymes and bacterial efflux pumps are successful antibiotic defence strategies in bacteria is shown in FIG. 3²⁹. The increase of resistance in Gram-negative bacteria in particular is a major cause for concern³⁹⁻³¹, as many Gram-negatives cause serious infections, such as pneumonia. Only a few antibiotics effective against Gram-negatives have been developed due to their innate defence mechanisms including low outer membrane permeability and high number of efflux pumps. Thus, with the rise of DR, many infections caused by Gram-negative bacteria have become untreatable³². Strategies that are able to quantify the efflux of agents from bacterial cells, especially Gram-negatives, for the assessment of potential new and reliable antimicrobial candidates would be very useful.

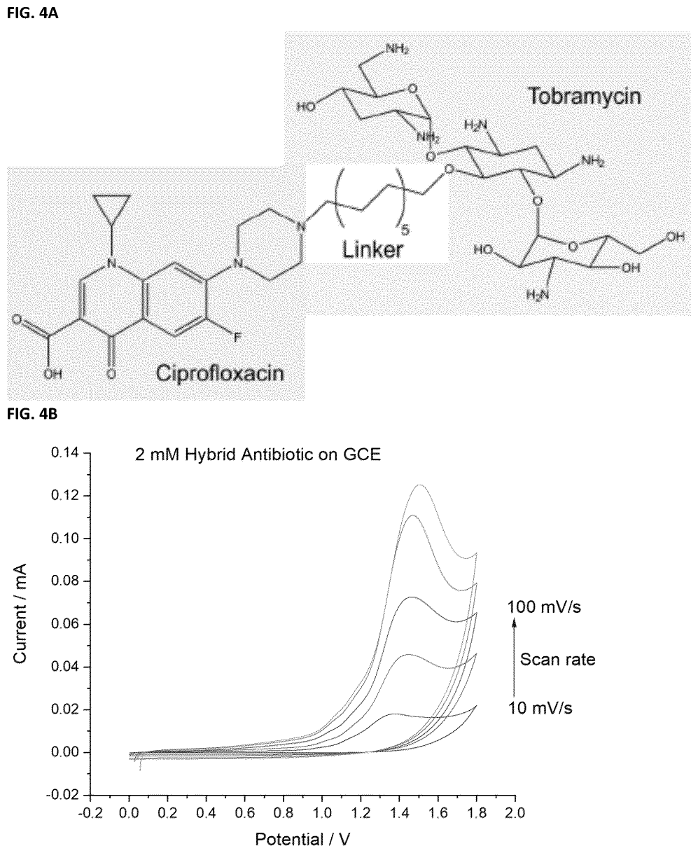

Most recently we have collected preliminary data about the electrochemical behavior of the antibiotics tobramycin and ciprofloxacin, which are in agreement with literature and show that both species can serve as potential efflux indicators. In addition, we have conducted first experiments on the antibiotic hybrid molecule tobramycin-ciprofloxacin (Tob-Cip, FIG. 4A). CVs show two irreversible peaks that can be assigned to the individual ciprofloxacin and tobramycin components at a potential of about 1.1 V and 1.3 V vs standard calomel reference electrode (FIG. 4B). As the ciprofloxacin peak is partially covered by the electrochemical response of tobramycin, we are currently working on electrode modifications using nanomaterials to separate the individual peaks more prominently. Nanoparticles have been successfully used in the literature to increase the surface area of electrodes to ultimately lowering the necessary overpotential applied to drive the oxidation/reduction reaction²⁶. These are encouraging first results, because it demonstrates that we can recognize and quantify antibiotic hybrids at electrodes. Other alternative experimental hybrid antibiotics, are to be tested. In addition, conventional electroactive antimicrobial agents that are known to be expelled by E. coli, such as ampicillin, and amoxicillin²⁸, will be characterized, resulting in a broad library of electroactive antibiotic substrates. Next to diffusional and thermodynamic parameters, the reversibility of the antibiotic redox reactions and whether the oxidized and reduced forms are stable may be assessed, as well as the possible occurrence of electrode fouling over multiple cycles. The term electrode fouling describes the blockage of the electroactive surface area of the electrode due to the absorption of solution species. These decomposition products can be characterized post-experimentally by X-ray photoelectron (XPS) spectroscopy or scanning electron microscopy (SEM). To avoid electrode fouling, testing may be restricted (e.g. avoiding sweeping over several electron-transfer reaction steps), or oxidative cleaning may be applied in between measurements to remove adsorbed material from the electrode surface. Also, thorough polishing of the electrode after each measurement using a water-alumina mix on micro-cloth polishing pads will assure the complete removal of possible reaction products may be useful.

The CV shape may be studied by finite element modelling, for example using a known approach²⁷, the physical processes may be described by a mass transport equation, the Butler-Volmer surface electron transfer kinetics, and chemical reaction kinetics in solution in a one-dimensional system. The output of this simulation is a CV current response, which may be fitted to the experimental CV. Thusly, redox reactions may be simulated to determine the Butler-Volmer kinetic parameters of antibiotics oxidation and reduction at the macro-electrode and a fitting of the concentration independent heterogeneous standard electrochemical rate constant as well as the standard electrode potential may be conducted. Accordingly, the reaction parameters for non-trivial redox systems may be determined, i.e. those that exhibit slow or asymmetric electron transfer kinetics, or irreversible side reactions. Such determined electrochemical reaction parameters might be useful for the drug efflux quantification, by choosing electrode potential, concentration range, and electrode material.

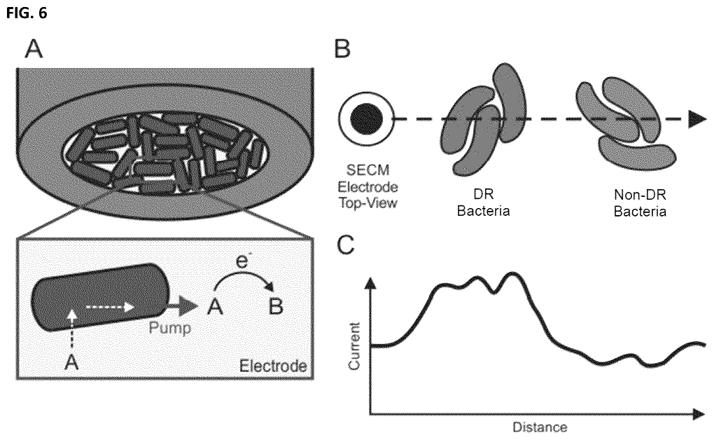

Bacteria, such as E. coli or P. aeruginosa would be useful as model organisms, and may be used in a buffer solution or may be drop-casted onto a macro- or microelectrode²³. These organisms have been shown to exhibit drug resistance associated with efflux pumps³³ and are relatively easy to handle with high proliferation rates. In addition, the importance of E. coli as contaminant in the food industry, as well as both bacteria types' impact in the medical sector make these organisms interesting targets. In both cases, bacteria may be exposed to electroactive antimicrobial agents that are known to be expelled by E. coli, such as amoxicillin^{34,36}, followed by an incubation period during which the agent may be taken up by the bacteria. Exact drug concentrations and incubation times would be evaluated. The exchange of solution to a fresh, drug-free buffer, would then allow for measurement of expelled drug molecules from the bacteria. For this purpose, the electrode may be biased at a potential far exceeding the formal potential of the antibiotic to drive the chemical reaction at the electrode surface (FIG. 6A). The conversion of any trace amounts of drugs, released by the bacteria, may be recorded through the potentiostat. Bacterial strains, which are non-resistant, DR, and deficient in efflux pump expression, such as P. aeruginosa (i.e. PAO 200 and PAO 750) are useful control organisms to assess the quantification method. As current approaches rely on complicated and costly methodologies³⁵, such as synchrotron based spectromicroscopy, the recognition of drug efflux by electrochemistry would allow for simple and direct measurement of antibiotic mass flux at low cost.

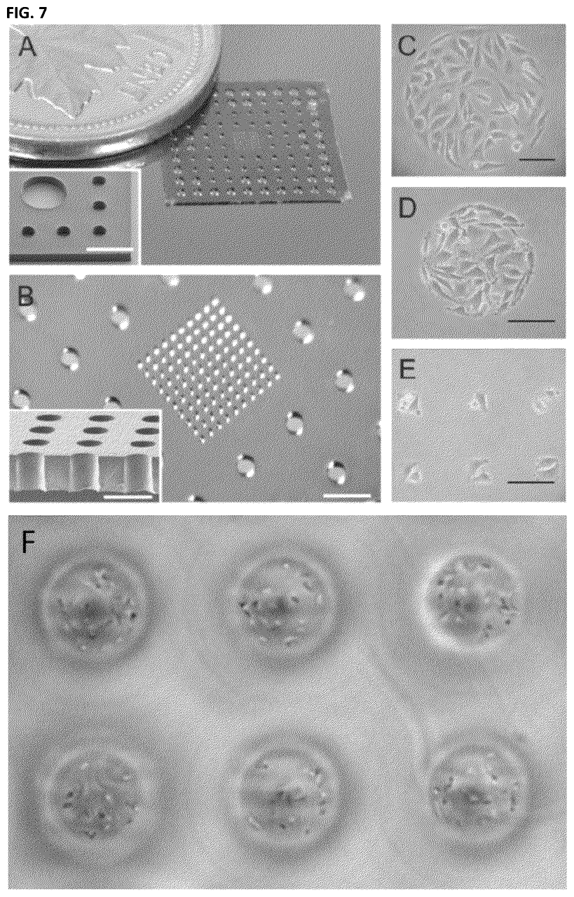

Target bacteria may be patterned in discrete locations on a substrate surface, for example using elastomeric through-hole membranes¹⁶. The applicability of these substrates has been demonstrated in the past for mammalian cancer cells (FIGS. 7A-E)¹⁶ and most recent preliminary data shows that this approach can be transferred to bacteria (FIG. 7F). For example, membranes for bacteria attachment may be an elastomeric polymer synthesised and masked into the defined membranes as shown in FIGS. 7A and B. Thereby, the hole-shape and -size may be modified and prepared according the bacteria to be immobilization (for example, 20 μm to 50 μm). The precise positioning of target bacteria onto plastic or glass substrates may be achieved by oxygen plasma treatment of the membranes placed on plastic or glass surfaces. The exposure to oxygen plasma renders the surface hydrophilic, promoting cell adhesion and will thereby allow for SECM studies on small islands of bacteria cultures or even single cells, whereby the drug resistant phenotype of these target cultures will be quantified. When bacteria are patterned on glass, a microelectrode may be brought in close proximity to the bacteria using the SECM setup to maximize the recorded current response. For this purpose, an approach curve over or next to a monolayer of bacteria may be carried out in the presence of a redox mediator, which is cell impermeable and will have no influence on the biological sample of interest.

Hexaammineruthinium(III)chloride (Ru(NH₃)₆Cl₃) is used in the literature¹⁶ for an approach curve. During an approach curve, while applying a reductive potential for Ru(NH₃)₆Cl₃, the microelectrode moves vertically towards the bacteria while recording the current. When the diffusion of the redox species gets hindered by the presence of the bacteria, the current value decreases and the motion of the electrode is stopped when it comes into contact with the bacteria. The probe may then be retracted to any desired distance above the bacteria (for example, 5 μm). Cell patterning is significant for Bio-SECM studies, as target bacteria can be easily located through the SECM-integrated optical microscope and bacteria will not be able to “crawl” away during repeated measurements. The SECM electrode may be moved horizontally across an island or single bacteria while recording the electrochemical current, resulting from the oxidation or reduction of a selected antimicrobial agent, exposed to the bacteria previously (FIGS. 6B and C). This allows for comparisons of bacteria of different resistance phenotypes, patterned in co-culture, at the same time and under the same conditions. Organisms of higher magnitude of DR (higher efflux rate) are thereby expected to result in higher current values during SECM measurements.

Due to the sensitivity of the SECM methodology it may be possible to tell the exact number of drug molecules exported from a single cell per second and per single cell. This would provide a numerical measure for the DR phenotype for any bacteria strain of interest. Although, E. coli may be employed as model organism the methods may be adapted to many different bacterial strains, such as Pseudomonas, will be measured and compared for their resistant phenotype by SECM. Drug efflux pump inhibitors, such as 3-(3′,4:5′-trimethoxyphenyl)-4,5,6-trimethoxyindanone-111, may be employed to analyse the sensitivity of the proposed method.

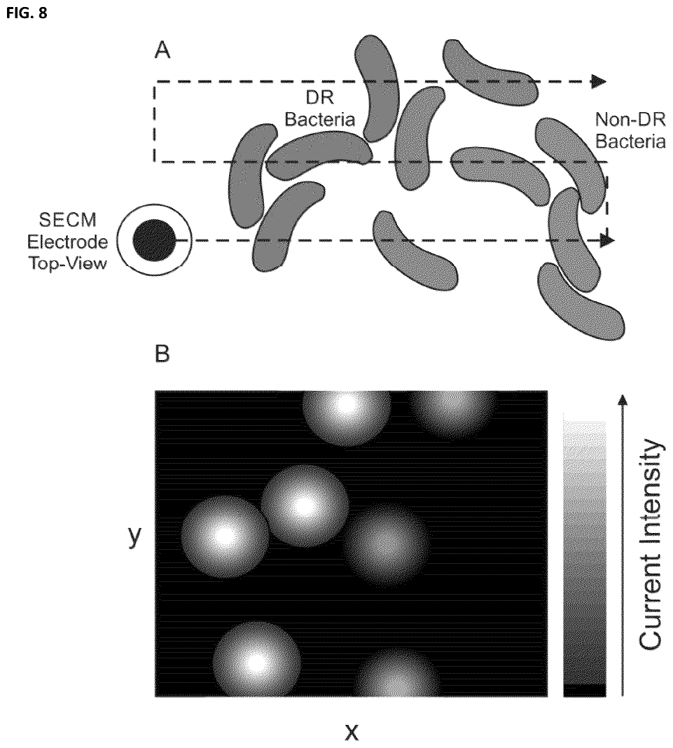

It is known that some DR bacteria have the ability to pass on their resistance to neighbouring bacteria³⁷. Understanding this phenomenon is key for developing models of DR progression across populations and may be investigated by electrochemistry, and specifically by SECM. DR and non-resistant bacteria may be patterned in close proximity to each other and the electrochemical current response to antibiotic treatment may be monitored in both populations over time. DR bacteria will be co-patterned in direct contact or any desired distance with non-resistant bacteria, employing the elastomeric through-hole membranes described above. Parts of the oxygen plasma treated surface may be covered by a commercially available elastomeric polymer (for example, polydimethylsiloxane (PDMF)), during cell exposure. The PDMF layer may then be removed and a second bacterial strain may be added. Bacterial strains may then be distinguished in co-cultures by fluorescently labelling their cytoplasmic membranes using different dye solutions. Combined fluorescent imaging and SECM would allow for the identification of various cell populations, even during cell proliferation or cell movement. The electrochemical current response to antibiotic treatment may be monitored simultaneously in populations and recorded over time for all bacteria. A change in current, as schematically shown in FIG. 8, may indicate an adaptation of non-resistant bacteria to the antibiotic in the presence of DR organisms. How quickly various bacteria strains can adopt antibiotic resistance depending on dosage, exposure time and nature of an antibiotic may be tested using this methodology. Different genetic models of bacteria may be monitored across populations and bacterial strains. Furthermore, the methods described herein may be used to test new antibiotic candidates, such as the Tob-Cip hybrid, and DR inhibitors may be tested and their local effect on a fraction of a population, as well as its influence on organisms within the same population, but in locally different areas. Quantitative measurements of the adaptation/transfer of DR properties between populations, may be subsequently used to establish models of DR progression. Monitoring DR initiation and progression quantitatively by electrochemistry may enable the establishment of DR population models based on reliable empirical data. Ultimately, gaining understanding of the development and spread of DR across organisms would greatly support efforts at developing strategies against this exceptional medical challenge.

Ciprofloxacin resistance is increasingly spread among infections and various pathogens exhibit resistance against this antibiotic. Pseudomonas aeruginosa cultures were analyzed to demonstrate the quantification of drug uptake in bacteria by electrochemistry. Two bacterial strains, one ciprofloxacin-susceptible (PA01) and one ciprofloxacin-resistant (PA262) strains were used for the experiments. The PA262 strain exhibits an overexpression of efflux systems, which expel antibiotics from the cell's cytosol to the exterior environment. This mechanism causes a decreased susceptibility against ciprofloxacin and makes these cultures resistant to the antibiotic.

Electrochemical detection by differential pulse voltammetry (DPV) of antibiotic uptake by Pseudomonas aeruginosa is shown for Ciprofloxacin (Cip) in FIG. 10. There were two controls, containing PBS solution containing 1 mM ciprofloxacin: Vial 1 (black) shows a peak current of approximately 55 μA prior to incubation; and Vial 2 (broken black) shows the current response of ciprofloxacin after 25 minutes of incubation at 37° C. These two controls demonstrate that the incubation at 37° C. does not lead to a degradation of ciprofloxacin in solution. Vials 3 and 4 (grey and broken grey) each contain P. aeruginosa cells in 1 mM Ciprofloxacin, where DPV measurements of the collected supernatant, whereby any ciprofloxacin taken up by the bacteria during the incubation time would be removed from the solution. Importantly, DPV measurements performed in the supernatant result in a lower current signal (FIG. 10, grey and broken grey) compared to the controls, indicating the uptake of ciprofloxacin by the bacteria. This preliminary data demonstrates that electrochemistry can detect drug uptake in bacteria. No statistically significant difference was observed between PA01 and PA262 bacteria strains. This indicated that the resistance mechanism towards ciprofloxacin in these cultures is not due to an inhibited drug uptake.

The uptake of another antibiotic, Tobramycin (2 mM) was analyzed in Pseudomonas aeruginosa using DPV. As shown in FIG. 11, currents recorded in control solutions (B and C) before and after incubation at 37° C. do not vary, indicating stable 2 mM Tobramycin concentrations in both control samples. As summarized in TABLE 3, the concentration of the control remains stable over different times of incubation at 37° C. As shown in TABLE 3, the wild type strain PAO1 takes up approximately 20% of Tobramycin from the solution at incubation times of 15 and 30 min, indicating a rapid (<15 min) establishment of equilibrium of intracellular and extracellular Tobramycin. At 15 min, the hyper-susceptible PAO750 removes about 26% of Tobramycin, which is slightly more than the wild type. This may be due to the absence of efflux pumps on the cell membranes, so that bacteria do not have the opportunity to expel parts of the internalized Tobramycin back into solution. At 30 min, we see that the uptake is failing, probably due to cell lysis of PAO750. A concentration of 2 mM Tobramycin appears to have been too high for the cells to withstand. A similar effect is seen in PAO1 at an incubation time of 60 min. As both of these strains are not resistant to Tobramycin, cell lysis at prolonged incubation times was expected.

TABLE 3
Percentage of tobramycin left in the supernatant
after various incubations.

| Incubation | | | |
|---|---|---|---|
| Time | Percentage of Tobramycin in Supernatant^{a, b} | | |
| (minutes) | Control^{c} | PAO1 | PAO750 |
| 15 | 99% | 80% | 74% |
| 30 | 99% | 81% | 95% |
| 60 | 95% | 99% | 99% |

^{a}Cells were incubated with 2 mM Tobramycin in 1X PBS, 0.4% glucose at 37° C. with aeration;
^{b}Remaining Tobramycin was measured using DPV on GCE v. Ag/AgCl at pH 3;
^{c}Control was 2 mM Tobramycin in 1X PBS, 0.4% glucose without bacterial cells.

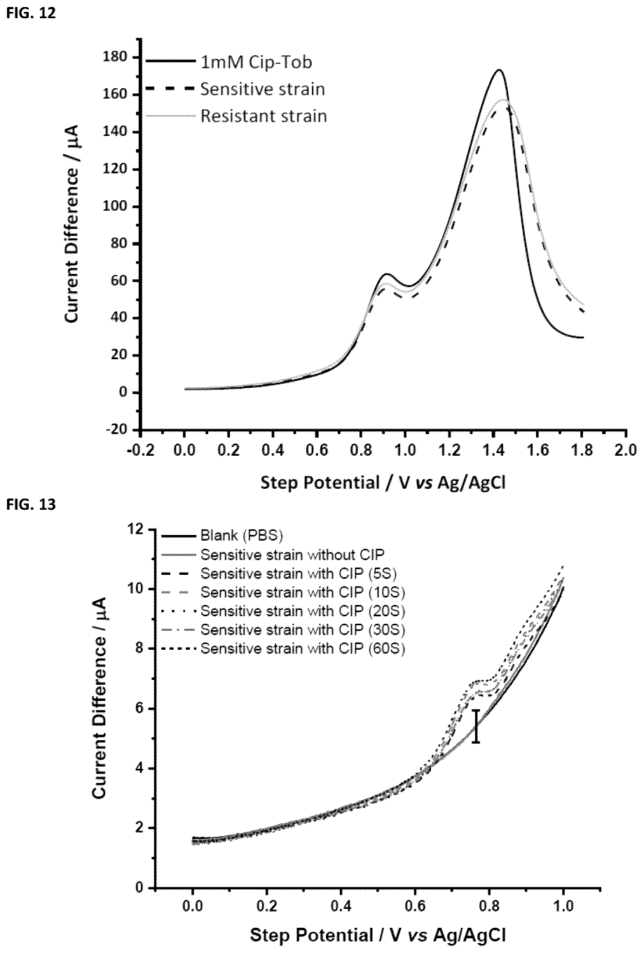

The ability of cells to take up a newly developed antibiotic hybrid was also evaluated (see FIG. 12). A Ciprofloxacin-Tobramycin (Cip-Tob) hybrid influx was monitored in P. aeruginosa by DPV. This hybrid was specifically developed to overcome resistance against ciprofloxacin in pathogens. The tobramycin moiety facilitates the uptake of the molecule, whereas the ciprofloxacin moiety, once inside the bacteria is expected to kill the pathogenic bacteria. A recent publication by the inventors further characterizes this hybrid antibiotic by electrochemistry⁴⁷.

FIG. 12 shows DPV measurements of the Cip-Tob hybrid prior to exposure to bacteria (black) and after incubation with PAO1 (broken black) and PAO750 (grey). Two pronounced peaks can be observed, representing the ciprofloxacin and tobramycin molecules as shown. Looking at the Tobramycin peak, the uptake of the drug by the bacteria becomes obvious. As expected, no significant difference between the bacterial strains was observed, as both strains are not resistant to Tobramycin and the resistance mechanism is based on the efflux of drugs in these bacteria.

To test the efflux of ciprofloxacin, Pseudomonas bacteria were incubated in a solution of PBS and ciprofloxacin. After 25 min of incubation, the bacteria suspension is centrifuged, cells are resuspended in PBS and drop-casted onto a 3-mm glassy carbon macroelectrode (see FIG. 1D). Other macroelectrode materials, such as gold, platinum, etc. could be employed in the same way. The electrode is then placed in a KCl solution and DPV is performed at a potential rage of zero to 1.0V. This potential range would be adjusted depending on the drug of interest. Ciprofloxacin oxidizes at a potential of approximately 0.7V vs Ag/AgCl reference electrode. A current increase is only expected, if the bacteria are exporting ciprofloxacin, which is then oxidized at the electrode.

FIG. 13 shows DPV measurements in the absence and presence of bacteria at the electrode. Two controls are shown. A blank (black) demonstrates the current profile in the absence of both bacteria and ciprofloxacin. No current peak is observed, as there is no ciprofloxacin present in solution. A second control (grey) shows drop-casted bacteria do not result in a current increase, if they were not incubated in ciprofloxacin. The error bar indicates the experimental error and variations in the controls. After incubation with ciprofloxacin, drop-casted bacteria result in a significant increase in current due to the export of ciprofloxacin from the bacteria, as shown in the various dotted and dashed curves in FIG. 13. When the electrode is placed in the KCl solution, various time intervals were applied before driving the oxidation reaction at the electrode. This gives the bacteria different time intervals to export ciprofloxacin to the cell exterior and demonstrate that a longer wait time results in a higher current peak.

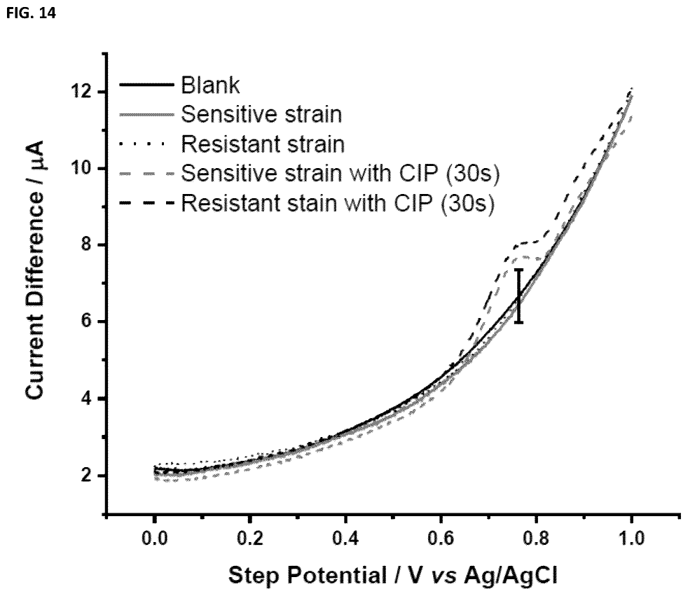

To differentiate between ciprofloxacin-resistant and -susceptible bacteria, PAO1 and PA262 Pseudomonas strains were drop-casted individually at macro-electrodes. As shown in FIG. 14, a significant current increase is observed with both species, whereby the resistant strain appears to result in a higher current than the susceptible strain, probably due to an enhances efflux of ciprofloxacin. The experiments shown herein suggest that electrochemistry is able to detect the uptake and efflux of antibiotics and chemotherapeutics.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings.

### REFERENCES

1 X. Lv, W. Ge, Q. Li, Y. Wu, H. Jiang, and X. Wang, ACS Appl. Mater. Interfaces, 6, 11025-11031 (2014).
2 World Health Organization. Antimicrobial Resistance—Fact Sheet; (2018). https://www.who.int/news-room/fact-sheets/detail/antimicrobial-resistance. Accessed Nov. 9, 2019.
3 World Health Organization. Global action plan on antimicrobial resistance; (2015). https://www.who.int/antimicrobial-resistance/global-action-plan/en/. Accessed Nov. 9, 2019.
4 Government of Canada. Government of Canada's response to antimicrobial resistance; (2018). https://www.canada.ca/en/public-health/services/antibiotic-antimicrobial-resistance/government-canada-response-antimicrobial-resistance.html. Accessed Nov. 9, 2019.
5 J. Sun, A. R. Warden, J. Huang, W. Wang, and X. Ding, Anal. Chem., 91, 7524-7530 (2019).
6 R. Article, J. Pathol., 205, 275-292 (2005).
7 H. Nikaido, Annu. Rev. Biochem., 78, 119-146 (2009).
8 S. N. Aleksakhina, A. Kashyap, and E. N. Imyanitov, Biochim. Biophys. Acta—Rev. Cancer, 1872, 188310 (2019).
9 G. Housman, S. Byler, S. Heerboth, K. Lapinska, M. Longacre, N. Snyder, and S. Sarkar, Cancers (Basel)., 6, 1769-1792 (2014).
10 G. Divyapriya, P. Thangadurai, and I. Nambi, ACS Sustain. Chem. Eng., 6, 3453-3462 (2018).
11 A. A. Ensafi, A. R. Allafchian, and R. Mohammadzadeh, Anal. Sci., 28, 705-710 (2012).
12 H. Yi and C. Li, Russ. J. Electrochem., 43, 1377-1381 (2007).
13 L. Fotouhi and M. Alahyari, Colloids Surfaces B Biointerfaces, 81, 110-114 (2010).
14 C. E. Banks, T. Killard, and B. J. Venton, Anal. Methods, 11, 2736-2737 (2019).
15 S. Kuss, R. Cornut, I. Beaulieu, M. A. Mezour, B. Annabi, J. Mauzeroll, Assessing multidrug resistance protein 1-mediated function in cancer cell multidrug resistance by scanning electrochemical microscopy and flow cytometry, Bioelectrochemistry. 82 (2011)
16 S. Kuss, D. Polcari, M. Geissler, D. Brassard, J. Mauzeroll, Assessment of multidrug resistance on cell coculture patterns using scanning electrochemical microscopy, Proc. Natl. Acad. Sci. U.S. A. 110 (2013) 9249-9254.
17 S. Kuss, D. Trinh, L. Danis, J. Mauzeroll, High-Speed Scanning Electrochemical Microscopy Method for Substrate Kinetic Determination: Method and Theory, Anal. Chem. 87 (2015) 8096-8101.
18 S. Kuss, D. Trinh, J. Mauzeroll, High-Speed Scanning Electrochemical Microscopy Method for Substrate Kinetic Determination: Application to Live Cell Imaging in Human Cancer, Anal. Chem. 87 (2015) 8102-8106.
19 L. Danis, D. Polcari, A. Kwan, S. M. Gateman, J. Mauzeroll, Fabrication of Carbon, Gold, Platinum, Silver and Mercury Ultramicroelectrodes with Controlled Geometry, Anal. Chem. 87 (2015) 2565-2569.
20 A. J. Bard, M. V Mirkin, Scanning Electrochemical Microscopy, Second, Marcel Decker Inc., USA: New York, 2001.
21 J. Mauzeroll, S. B. Schougaard, Electrochemical Microscopy of Living Cells, Scanning Electrochem. Microsc. (2012) 379.
22 S. Bergner, J. Wegener, F.-M. Matysik, Monitoring passive transport of redox mediators across a confluent cell monolayer with single-cell resolution by means of scanning electrochemical microscopy, Anal. Methods. 4 (2012) 623-629.
23 S. Kuss, E. E. L. Tanner, M. Ordovas-Montanes, R. G. Compton, Electrochemical recognition and quantification of cytochrome c expression in: Bacillus subtilis and aerobe/anaerobe Escherichia coli using N, N, N ‘, N’-tetramethyl-para-phenylene-diamine (TMPD), Chem. Sci. 8 (2017).
24 I. Beaulieu, S. Kuss, J. Mauzeroll, M. Geissler, Biological scanning electrochemical microscopy and its application to live cell studies, Anal. Chem. 83 (2011) 1485-1492.
25 R. G. Compton, C. E. Banks, Understanding voltammetry, 2nd edition, 2010.
26 X. Luo, A. Morrin, A. J. Killard, M. R. Smyth, Nanoparticles in Electrochemical Biosensors, in: H. S. Nalwa (Ed.), Encycl. Nanosci. Nanotechnol., American Scientific Publishers, 2011.
27 S. Kuss, R. G. Compton, Electrocatalytic detection of ascorbic acid using N,N,N′,N′ tetramethylpara-phenylene-diamine (TMPD) mediated oxidation at unmodified gold electrodes; reaction mechanism and analytical application, Electrochim. Acta. 242 (2017).
28 J. A. Delmar, C.-C. Su, E. W. Yu, Bacterial Multidrug Efflux Transporters, Annu. Rev. Biophys. 43 (2014) 93-117.
29 H. Nikaido, Multidrug resistance in bacteria, Annu. Rev. Biochem. 78 (2009) 119-146.
30 L. L. Silver, A Gestalt approach to Gram-negative entry, Bioorg. Med. Chem. 24 (2016) 6379-6389.
31 I. Karaiskos, H. Giamarellou, Multidrug-resistant and extensively drug-resistant Gram-negative pathogens: Current and emerging therapeutic approaches, Expert Opin. Pharmacother. 15 (2014) 1351-1370.
32 A. Rodrigo-Troyano, O. Sibila, The respiratory threat posed by multidrug resistant Gram negative bacteria, Respirology. 22 (2017) 1288-1299.
33 H. W. Boucher, G. H. Talbot, J. S. Bradley, J. E. Edwards, D. Gilbert, L. B. Rice, M. Scheid, B. Spellberg, J. Bartlett, Bad Bugs, No Drugs: No ESKAPE! An Update from the Infectious Diseases Society of America, Clin. Infect. Dis. 48 (2009) 1-12.
34 F. O. Olorunmola, D. O. Kolawole, A. Lamikanra, Antibiotic Resistance and Virulence Properties in Escherichia Coli Strains from Cases of Urinary Tract Infections, African J. Infect. Dis. 7 (2013) 1-7.
35 B. Cinquin, L. Maigre, E. Pinet, J. Chevalier, R. A. Stavenger, S. Mills, M. Réfrégiers, J.-M. Pages, Microspectrometric insights on the uptake of antibiotics at the single bacterial cell level, Sci. Rep. 5 (2015) 17968.
36 M. Fouladgar, M. R. Hadjmohammadi, M. A. Khalilzadeh, P. Biparva, N. Teymoori, H. Beitollah, Voltammetric Determination of Amoxicillin at the Electrochemical Sensor Ferrocenedicarboxylic Acid Multi Wall Carbon Nanotubes Paste Electrode, Int. J. Electrochem. Sci. 6 (2011) 1355-1366.
37 Centers for Disease Control and Prevention, Antibiotic/Antimicrobial Resistance, (2017). https://www.cdc.gov/drugresistance/about.html (accessed Oct. 11, 2017).
38 Albrecht, T. et al. Curr Opin Electrochem 2018. 7, 138.
39 Stevenson, K. J. et al. Curr Opin Electrochem 2017. 6(1), 38.
40 F.-R. F. Fan, J. Fernandez, B. Liu, J. Mauzeroll, C. G. Zoski, Platinum and gold inlaid disks≥5 μm diameter, in: C. G. Zoski (Ed.), Handbook of Electrochemistry, Elsevier B. V, Amsterdam, 2007, pp. 189-199.
41 J. Mauzeroll, R. J. LeSuer, Laser-pulled ultramicroelectrodes, in: C. G. Zoski (Ed.), Handbook of Electrochemistry, Elsevier B. V, Amsterdam, 2007, pp. 199-211.
42 Hatamluyi B. et al. Biosens Bioelectron. (2018) “Au/Pd@rGO nanocomposite decorated with poly (L-Cysteine) as a probe for simultaneous sensitive electrochemical determination of anticancer drugs, Ifosfamide and Etoposide” 120:22-29.
43 Suhito, I. R. et al. Sensors and Actuators B: Chemical (2019) “Rapid and sensitive electrochemical detection of anticancer effects of curcumin on human glioblastoma cells” 288: 527-534.
44 Hatamluyi B. et al. Sensors and Actuators B: Chemical (2020-available online 20 Dec. 2019) “A novel molecularly imprinted polymer decorated by CQDs@HBNNS nanocomposite and UiO-66-NH2 for ultra-selective electrochemical sensing of Oxaliplatin in biological samples” 307:127614.
45 Luu et al. Journal of Electroanalytical Chemistry Electrochemical characterization of carboplatin at unmodified platinum electrodes and its application to drug consumption studies in ovarian cancer cells 872 (2020) 114253.
46. Kuss S. et al. Forced Convection during Scanning Electrochemical Microscopy Imaging over living cells: Effect of Topographies and Kinetics on the Microelectrode Current. Electrochimica Acta, 2013, 110, 42. DOI: 10.1016/j.electacta.2013.03.149.
47. Islam M. R. et al. Electrochemical Characterization of the Antibiotic Hybrid Ciprofloxacin-Tobramycin. Electrochemistry Communications 2020, 119C, 106825. DOI: 10.1016/j.elecom.2020.106825.

What is claimed is:

1. A method of determining membrane permeability of a cell to a drug, the method comprising: (a) obtaining a biological sample; (b) dispersing at least one cell from the biological sample to a discrete location or attached to a discrete substrate; (c) exposing the at least one cell to one member of a drug panel in a drug solution, wherein the drug panel is comprised of drugs of a given concentration; (d) incubating the at least one cell from the biological sample in the drug for a given time; (e) obtaining at least one electro-analytical measurement of the discrete location adjacent the at least one cell.

2. The method of claim 1, wherein the method further comprises exchanging the drug solution for a drug-less solution.

3. The method of claim 2, wherein the method further comprises incubating the at least one cell from the biological sample in the drug-less solution for a given time.

4. The method of claim 3, wherein the method further comprises obtaining at least one further electro-analytical measurement of the discrete location adjacent to the at least one cell.

5. The method of any one of claims 1-4, wherein the drug is an electro-active drug.

6. The method of any one of claims 1-4, wherein the drug is selected from: an antibiotic drug and an anticancer drug.

7. The method of claim 6, wherein the antibiotic drug is selected from one or more of the following: ampicillin; penicillin; amoxicillin; neomycin; tobramycin; ciprofloxacin; levofloxacin; norfloxacin; enrofloxacin; ofloxacin; linezolid; tetracycline; and azithromycin; or a hybrid of two or more antibiotic drugs.

8. The method of claim 6, wherein the anticancer drug is selected from one or more of the following: 6-mercaptopurine; 5-fluorouracil; gemcitabine; doxorubicin; mitoxantrone; epirubicin; daunorubicin; valrubicin; cisplatin; temodal; oxaliplatin; carboplatin; etoposide; ifosfamide; erlotinib; irinotecan; and roscovitine; or a hybrid of two or more anti-cancer drugs.

9. The method of any one of claims 1-8, wherein the drug panel is comprised of multiple drugs each at a variety of concentrations or combinations of drugs each at a variety of concentrations.

10. The method of any one of claims 1-9, wherein the biological sample comprises bacteria isolated from a patient.

11. The method of any one of claims 1-9, wherein the biological sample comprises a cancer biopsy from a patient.

12. The method of any one of claims 1-11, wherein the electro-analytical measurement is made by one or more of the following: linear sweep voltammetry (LSV); cyclic voltammetry (CV); differential pulse voltammetry (DPV); differential pulse anodic stripping voltammetry (DPASV); square wave voltammetry (SWV); adsorptive stripping linear sweep voltammetry (AdSLSV); electrochemical impedance spectroscopy (EIS); chronoamperometry (CA); chronopotentiometry (CP); chronocoulometry (CC); impact chemistry (IC); scanning ion conductance microscopy (SICM); scanning electrochemical cell microscopy (SECCM); scanning photoelectrochemical microscopy (SPECM); and scanning electrochemical microscopy (SECM).

13. The method of any one of claims 1-12, wherein electrode is optimized for the electro-active drug or electro-active drugs at the discrete location.

14. An apparatus, the apparatus comprising (a) cell retention array having a plurality of array locations; and (b) a corresponding electrode array, wherein each electrode corresponds to each array location or a group of electrode locations and wherein the electrode is selected to be operable for a corresponding drug solution.

15. A microfluidic device, the microfluidic device comprising (a) a plurality of cell retention locations; and (b) a corresponding electrode for each cell retention location or locations and wherein the electrodes are selected to be operable for a corresponding drug solution which might be delivered to the retention location or locations.

16. The microfluidic device of claim 15, wherein the microfluidic device further comprises a system for fluid exchange at one or more of the retention locations.\

17. An apparatus, the apparatus comprising (a) a plurality of cell retention substrates; and (b) a corresponding electrode associated with each cell retention substrate, wherein the electrode is selected to be operable for a corresponding drug solution.

18. The apparatus of claim 17, wherein the cell retention substrates are beads.

19. An apparatus, the apparatus comprising:
a cell retention array having a plurality of array locations;
wherein each cell retention array location corresponds to an electrode, and wherein each electrode is suitable for deposition of a cell on the surface of the electrode.

20. The apparatus of claim 19, wherein each electrode is operable to retain the cell on the electrode by dropcasting.

21. The apparatus of claim 20, wherein the each electrode is operable to receive a drug solution.

22. A method of determining membrane permeability of a cell to a drug, the method comprising: (a) obtaining a biological sample; (b) dispersing at least one cell from the biological sample to a surface or attached to a surface; (c) exposing the at least one cell to a drug solution, wherein the drug solution has a given drug concentration; (d) incubating the at least one cell from the biological sample in the drug solution for a given time; (e) obtaining at least one electro-analytical measurement of the at least one cell by impact chemistry (IC), whereby the at least one cell from the biological sample is made to collide with an electrode.

23. The method of claim 22, wherein the method further comprises exchanging the drug solution for a drug-less solution.

24. The method of claim 23, wherein the method further comprises incubating the at least one cell from the biological sample in the drug-less solution for a given time.

25. The method of claim 24, wherein the method further comprises obtaining at least one further electro-analytical measurement of the at least one cell from the biological sample.

26. The method of any one of claims 22-25, wherein the drug is an electro-active drug.

27. The method of any one of claims 22-26, wherein the drug is selected from: an antibiotic drug and an anticancer drug.

28. The method of claim 27, wherein the antibiotic drug is selected from one or more of the following: ampicillin; penicillin; amoxicillin; neomycin; tobramycin; ciprofloxacin; levofloxacin; norfloxacin; enrofloxacin; ofloxacin; linezolid; tetracycline; and azithromycin; or a hybrid of two or more antibiotic drugs.

29. The method of claim 27, wherein the anticancer drug is selected from one or more of the following: 6-mercaptopurine; 5-fluorouracil; gemcitabine; doxorubicin; mitoxantrone; epirubicin; daunorubicin; valrubicin; cisplatin; temodal; oxaliplatin; carboplatin; etoposide; ifosfamide; erlotinib; irinotecan; and roscovitine; or a hybrid of two or more anti-cancer drugs.

30. The method of any one of claims 22-29, wherein the dispersing of at least one cell from the biological sample to the surface or attached to the surface drug panel repeated on multiple discrete surfaces so that multiple drugs at a variety of concentrations or combinations of drugs each at a variety of concentrations are available for IC electro-analytical measurement.

31. The method of any one of claims 22-30, wherein the biological sample comprises bacteria isolated from a patient.

32. The method of any one of claims 22-30, wherein the biological sample comprises a cancer biopsy from a patient.

33. The method of any one of claims 22-32, IC electrode is a wire electrode.

34. The method of any one of claims 22-33, wherein electrode is optimized for the electro-active drug or electro-active drugs at the discrete location.
